(19) United States
(12) Patent Application Publication
(10) Pub. No.: US 2002/0176841 A1
(43) Pub. Date: Nov. 28, 2002

(54) Pharmaceutical formulations for sustained release

(75) Inventors: Nicholas Barker, Southborough, MA (US); Janet L. Wolfe, Newton, MA (US)

Correspondence Address: LAHIVE & COCKFIELD, 28 STATE STREET, BOSTON, MA, 02109, US

(73) Assignee: Praecis Pharmaceuticals Inc., Waltham, MA (US)

(21) Appl. No.: 10/102,530

(22) Filed: Mar. 19, 2002

Related U.S. Application Data
(60) Provisional application No. 60/277,195, filed on Mar. 19, 2001.

(51) Int. Cl. A61K 31/785

(52) U.S. Cl. 424/78.12

(57) ABSTRACT

Sustained delivery pharmaceutical compositions comprising a solid ionic complex of a pharmaceutically active compound and an ionic macromolecule are provided by the present invention. The pharmaceutical compositions of the invention allow for loading of high concentrations of pharmaceutically active compounds and for delivery of a pharmaceutically active compound for prolonged periods of time, e.g., one month, after administration. Methods for preparing these pharmaceutical compositions, as well as methods of using them to treat a subject are also provided.

### RELATED APPLICATIONS

This application claims priority to U.S. Provisional Patent Application Serial No. 60/277,195 filed Mar. 19, 2001, the entire contents of which are incorporated herein by reference.

### BACKGROUND OF THE INVENTION

An area of current research focus in the pharmaceutical industry is the development of methods for the controlled or sustained release of drugs. Such methods obviate certain problems associated with traditional methods for administering drugs, such as non-compliance of patients with a prescribed medication schedule, the need for frequent injections, and fluctuating concentrations of the drug in the body. Methods for sustained or controlled drug release typically utilize an implanted device, such as an osmotic pump, or a drug dispersed in a biocompatible polymer matrix, which can be implanted, administered orally or injected.

Attempts to develop sustained-release formulations have included the use of a variety of biodegradable and non-biodegradable polymer (e.g. poly(lactide-co-glycolide)) microparticles containing the active ingredient (see e.g., Wise et al. (1973) Contraception 8:227-234 and Hutchinson et al. (1985) Biochem. Soc. Trans. 13:520-523), and a variety of techniques are known by which active agents can be incorporated into polymeric microspheres (see, e.g., U.S. Pat. No. 4,675,189 and references cited therein).

The release characteristics for the active ingredient from microparticles prepared by methods such as those described above may be continuous or discontinuous, and in some cases, the initial level of active ingredient release is too high or too low.

Clearly the need still exists for an improved method for preparing pharmaceutical compositions containing an active ingredient, which method is simple, inexpensive, versatile, and, most importantly, which provides for high loading efficiencies and yields, thereby allowing for more consistent active ingredient release over an extended period of time.

### SUMMARY OF THE INVENTION

The present invention provides pharmaceutical compositions which are suitable for the sustained release of a pharmaceutically active compound in vivo, and to methods of producing such pharmaceutical compositions. The invention further relates to methods of administering a pharmaceutically active compound using these pharmaceutical formulations.

In one embodiment, the invention provides a solid ionic complex comprising an ionic carrier macromolecule and a pharmaceutically active compound. Preferably, the pharmaceutically active compound is non-peptidic and bears an electronic charge which is opposite in sign to the charge of the ionic macromolecule. In a preferred embodiment, the ionic macromolecule and the pharmaceutically active compound together form a solid ionic complex.

The ionic macromolecule can be a linear, branched or cross-linked polymer which bears a net positive or negative charge at a certain pH and the pharmaceutically active compound bears an electronic charge at the same pH which is opposite in sign to that of the ionic macromolecule. Preferably, the pharmaceutically active compound bears a charge of at least 2+, 3+, 4+, or 5+ or at least 2−, 3−, 4−, or 5− at the pH

In one embodiment, the pharmaceutically active compound may include at least one functional group selected from the group consisting of primary amino groups, secondary amino groups, tertiary amino groups, imino groups, quaternary ammonium groups, amidino groups, guanidino groups, phosphonium groups and sulfonium groups.

In another embodiment, the pharmaceutically active compound may include at least one functional group selected from the group consisting of carboxylate groups, sulfonate groups, phosphonate groups, sulfamate groups, sulfate ester groups, phosphate ester groups, sulfinate groups, phosphinate groups, carbonate groups, thiocarboxylate groups and carbamate groups.

The pharmaceutically active compound may have a molecular weight of about 1000 amu or less, about 900 amu or less, about 800 amu or less, about 700 amu or less, about 600 amu or less, about 500 amu or less, about 400 amu or less, about 300 amu or less, or about 200 amu or less.

In another embodiment, the ionic macromolecule may be a polypeptide or a polysaccharide. In yet another embodiment, the ionic macromolecule may comprise at least one functional group selected from the group consisting of carboxylic acid, sulfonic acid, sulfamic acid, primary amine, secondary amine, tertiary amine, quaternary ammonium, guanidino and amidino.

In a preferred embodiment, a single dose of the solid ionic complex provides sustained delivery of the pharmaceutically active compound to a subject for at least one, two, three, four or five weeks after the pharmaceutical composition is administered to the subject. The solid ionic complex may, for example, be a lyophilized solid or it may be suspended as a liquid suspension or dispersed as a semi-solid dispersion.

In a further embodiment, the pharmaceutically active compound content of the solid ionic complex is at least 50%, 55%, 60%, 65%, 70%, 75%, 80%, 85%, 90%, 95%, or 98% by weight. In another embodiment, the pharmaceutically active compound content of the solid ionic complex is 50% to 90% by weight, 40%-90% by weight, 40% to 80% by weight, or 60% to 95% by weight. Ranges of values using a combination of any of the above recited values as upper and/or lower limits are intended to be included.

In another embodiment, the pharmaceutically active compound and the ionic macromolecule used to form the solid ionic complex are combined at a weight ratio of ionic macromolecule:pharmaceutically active compound of 0.8:1 to 0.1:1. Ranges intermediate to the above recited values, e.g., 0.8:1 to 0.4:1, 0.6:1 to 0.2:1, or 0.5:1 to 0.1:1 are also intended to be part of this invention. Other possible ratios of ionic macromolecule:pharmaceutically active compound include 0.5:1, 0.4:1, 0.3:1, 0.25:1, 0.15:1, and 0.1:1. Moreover, ranges of values using a combination of any of the above recited values as upper and/or lower limits are intended to be included. In a preferred embodiment, the complex is not a microcapsule.

In another aspect, the present invention provides a packaged formulation for treating a subject for a condition treatable with a pharmaceutically active compound, which includes the pharmaceutical compositions of the invention packaged with instructions for using the compositions for treating a subject having a condition treatable with a pharmaceutically active compound.

In yet another aspect, the present invention provides a method for treating a subject for a condition treatable with a pharmaceutically active compound. The method includes administering to the subject the pharmaceutical compositions of the invention in an amount effective to treat the condition.

In a further aspect, the resent invention provides a method for preparing a pharmaceutical formulation of the invention. The method includes providing a pharmaceutically active compound and an ionic macromolecule; combining the pharmaceutically active compound and the ionic macromolecule under conditions such that a solid ionic complex of the pharmaceutically active compound and the ionic macromolecule forms; and preparing a pharmaceutical formulation comprising the solid ionic complex. The method may further include sterilizing the solid ionic complex by gamma irradiation or electron beam irradiation

In one embodiment, a solution, e.g., an aqueous solution, of the pharmaceutically active compound and a solution, e.g., an aqueous solution, of the ionic macromolecule are combined until a water-insoluble complex of the pharmaceutically active compound and the ionic macromolecule precipitates. In a preferred embodiment, the water-insoluble complex is formed using aseptic procedures.

Other features and advantages of the invention will be apparent from the following detailed description and claims.

### DETAILED DESCRIPTION OF THE INVENTION

The present invention provides pharmaceutical compositions suitable for the sustained release of a pharmaceutically active compound in vivo. The invention further provides methods of making and using the sustained release pharmaceutical compositions of the invention. The advantages of the pharmaceutical compositions of the invention include the ability for delivery of a pharmaceutically active compound, either systemically or locally, for prolonged periods (e.g., several weeks, one month or several months) and the ability to load high concentrations of the pharmaceutically active compound into the solid ionic complex that is formed.

In one embodiment, the invention provides a pharmaceutical composition for the sustained release of a pharmaceutically active compound. The composition comprises an ionic complex that includes a pharmaceutically active compound having a net electronic charge at a desired pH and an ionic carrier macromolecule. At the desired pH, the ionic macromolecule has a net electronic charge which is opposite in sign to the net electronic charge of the pharmaceutically active compound. The pharmaceutically active compound can bear a net positive charge or a net negative charge at a the desired pH. Preferably, the compound bears a net positive charge of 2+ or greater at the desired pH or a net negative charge of 2− or greater at the desired pH.

The pharmaceutically active compound can be any non-peptidic compound which forms a suitable solid ionic complex with a pharmaceutically acceptable ionic macromolecule. A “non-peptidic compound”, as defined herein, is a compound which includes no more than one peptide bond. Preferred non-peptidic compounds have a molecular weight of 1000 daltons or less, more preferably 750 daltons or less, and most preferably 500 daltons or less. Preferably, the pharmaceutically active compound is monomeric, i.e., not polymeric or oligomeric. A “monomeric compound”, as this term is used herein, does not comprise repeating structural units, for example, repeating backbone structural units. More preferably, the compound is a monomeric condensed compound. A “condensed compound”, as this term is used herein, is a compound having a structure with ten or fewer contiguous linear (unbranched) chemical bonds, i.e., a condensed compound has no more than ten contiguous linear bonds which do not define, or are not a part of, a cyclic structure. Preferably, a condensed molecule has nine, eight, seven, six, five or fewer contiguous linear chemical bonds. The cyclic structure is, preferably, a ten-membered monocyclic structure or smaller or a fused polycyclic structure. The cyclic structure can be aliphatic or aromatic, or, if polycyclic, a combination of aromatic and aliphatic.

Preferably, the pharmaceutically active compound has a net positive electronic charge of at least +1 or a net negative electronic charge of at least −1. As used herein, the term “electronic charge” refers to the greatest net electronic charge the molecule bears in the range of pH 5.0 to pH 9.0 (e.g., pH 5.0, pH 6.0, pH 7.0, pH 8.0, or pH 9.0). Preferably, the compound has a net electronic charge at physiological pH (e.g., pH 7.4). In a preferred embodiment, the pharmaceutically active compound has a net positive electronic charge of at least +2 or a net negative electronic charge of at least −2. Examples of suitable pharmaceutically active compounds include non-peptidic compounds having a molecular weight of about 1000 amu or less and a net charge of at least +1 or −1. Preferred pharmaceutically active compounds have a molecular weight of 750 amu or less, 600 amu or less or 500 amu or less and have net electronic charge of +1, +2, +3 or +4 or greater, or −1, −2, −3 or −4 or greater.

Examples of pharmaceutically active compounds that can be used in the pharmaceutical compositions of the invention include antitumor antibiotics, such as bleomycin, dactinomycin, actinomycin D, mitomycin and plicamycin; analgesics and andronergics, such as codeine, chlorpheniramine, hydrocodone, phenylephrine, dihydrocodeine, phenylpropanolamine, pseudoephedrine, dichloralphenazone, isometheptene, oxycodone, pentazocine, phenyltoloxamine, propoxyphene, pseudoephedrine, alfentanil, aspirin, orphenadrine, propoxyphene, carisoprodol, meprobamate, methocarbamol, atropine, hyoscyamine; methenamine, buprenorphine, butorphanol, celecoxib, clonidine, diclofenac, misoprostol, diflunisal, etodolac, fenoprofen, fentanyl, flurbiprofen, ibuprofen, hydromorphone, indomethacin, ketoprofen, ketorolac, levomethadyl, levorphanol, salicylic acid, meclofenamate, mefenamic acid, meperidine, promethazine, methadone, morphine, nabumetone, nalbuphine, naloxone, naproxen, oxaprozin, oxycodone, oxymorphone, phenazopyridine, sulfisoxazole, piroxicam, propoxyphene, salsalate, thiosalicylate, sufentanil, sulindac, tolmetin and tramadol.

Suitable pharmaceutically active compounds also include local anesthetics, such as antipyrine; benzocaine, butamben; tetracaine, bupivacaine, epinephrine, chloroprocaine, cocaine, dyclonine, etidocaine, proparacaine, lidocaine, prilocaine, mepivacaine, levonordefrin, procaine, proparacaine, ropivacaine and tetracaine.

Other suitable pharmaceutically active compounds include gastrointestinal agents, for example, difenoxin, hyoscyamine; phenobarbital, scopolamine, butabarbital, bethanechol, bisacodyl, chlordiazepoxide; clidinium, choline; dexpanthenol, cimetidine, cisapride, promethazine, dicyclomine, diltiazem, dimenhydrinate, diphenoxylate, docusate, dolasetron; dronabinol, droperidol, fentanyl, erythromycin, famotidine, glycopyrrolate, granisetron, pramoxine, lansoprazole, loperamide, mepenzolate, meperidine; mesalamine, 5-ASA, methscopolamine, metoclopramide, monoctanoin, nizatidine, olsalazine, omeprazole, ondansetron, orlistat, ochlorperazine, propantheline, ranitidine, sulfasalazine, thiethylperazine, trimethobenzamide, ursodeoxycholic acid, ursodiol; antipsychotic agents, such as amitriptyline; perphenazine, chlorpromazine, clozapine, fluphenazine, haloperidol, loxapine, mesoridazine, molindone, olanzapine, perphenazine, pimozide, prochlorperazine, promazine, quetiapine, risperidone, thioridazine, thiothixene, trifluoperazine, triflupromazine and zyprasidone; antimalarial agents, such as chloroquine, halofantrine, hydroxychloroquine, mefloquine, primaquine, pyrimethamine, pyrimethamine; sulfadoxine and quinine; antitussive agents, such as chlorpheniramine; dextromethorphan; guaifenesin; phenylpropanolamine, benzonatate, bromodiphenhydramine; brompheniramine; carbetapentane; carbinoxamine; and triprolidine; anticonvulsant agents, such as acetazolamide, carbamazepine, clonazepam, diazepam, ethosuximide, ethotoin, felbamate, fosphenytoin, gabapentin, lamotrigine, lorazepam, mephenytoin, mephobarbital, methsuximide, pentobarbital, phenobarbital, phenytoin, primidone, secobarbital, tiagabine, topiramate, valproic acid, divalproex; cholinesterase inhibitors, such as ambenonium, atropine; edrophonium, demecarium, donepezil, isoflurophate, neostigmine, physostigmine, pyridostigmine and tacrine; mydriatics, such as apraclonidine, atropine, cyclopentolate, homatropine, hydroxyamphetamine; tropicamide, scopolamine and sulfacetamide; sympathomimetics, such as acrivastine; albuterol, levalbuterol, amphetamine; dextroamphetamine, antazoline; naphazoline, antipyrine; apraclonidine, azatadine; benzphetamine, bitolterol, brompheniramine; bupivacaine; caramiphen; carbetapentane; carbidopa; levodopa, carbinoxamine; methscopolamine; phenindamine; phenyltoloxamine, iramine; pyrilamine, clemastine; triprolidine, dexbrompheniramine; dexchlorpheniramine; diethylpropion, dipivefrin, dobutamine, dopamine, dyphylline; hydroxyzine; isoetharine, isoproterenol, loratadine; mazindol, mephentermine, levonordefrin, methoxamine, midodrine, naphazoline, phendimetrazine, phentermine, pirbuterol, ritodrine, salmeterol, terbutaline, fonnoterol and tetrahydrozoline; antihypertensive agents, such as acebutolol, amiloride, amlodipine, benazepril, atenolol, atenolol; chlorthalidone, bendroflumethiazide; betaxolol, bisoprolol, bumetanide, candesartan, captopril, carteolol, carvedilol, chlorothiazide, chlorthalidone, clonidine, methyclothiazide, diazoxide, diltiazem, enalapril, doxazosin, enalaprilat, felodipine, epoprostenol, esmolol, ethacrynic acid, felodipine, fosinopril, furosemide, guanabenz, guanadrel, guanethidine, guanfacine, hydralazine, reserpine, irbesartan, labetalol, lisinopril, losartan, metoprolol, moexipril, reserpine, spironolactone, timolol, triamterene, valsartan, hydroflumethiazide, indapamide, isradipine, mecamylamine, methyclothiazide, metolazone, minoxidil, nadolol, nicardipine, nifedipine, nisoldipine, penbutolol, phenoxybenzamine, phentolamine, pindolol, polythiazide, prazosin, quinapril, ramipril, sotalol, telmisartan, terazosin, timolol, tolazoline, torsemide, trandolapril, verapamil and triamterene; antiarrhythmia agents, such as acebutolol, amiodarone, atenolol, bretylium, disopyramide, encainide, esmolol, flecainide, ibutilide, mexiletine, moricizine, phenytoin, procainamide, propafenone, quinidine, sotalol and tocainide; anti-obesity agents, such as sibutramine; anti-infective agents, such as abacavir, acyclovir, albendazole, amantadine, amikacin, aminosalicylic acid, amoxicillin, clavulanic acid, amphotericin B, ampicillin, sulbactam, atovaquone, azithromycin, aztreonam, bacampicillin, bacitracin, metronidazole, tetracycline, butenafine, butoconazole, capreomycin, carbenicillin, cefaclor, cefadroxil, cefamandole, cefazolin, cefdinir, cefepime, cefixime, cefmetazole, cefonicid, cefoperazone, cefotaxime, cefotetan, cefoxitin, cefpodoxime, cefprozil, ceftazidime, ceftibuten, ceftizoxime, ceftriaxone, cefuroxime, cephalexin, cephapirin, cephradine, chloramphenicol, chloroquine, chloroxine, ciclopirox, clioquinol, chlortetracycline, cidofovir, cinoxacin, ciprofloxacin, clarithromycin, clindamycin, clofazimine, clotrimazole, cloxacillin, colistimethate, colistin, crotamiton, cycloserine, dapsone, delavirdine, demeclocycline, dicloxacillin, didanosine, dirithromycin, doxycycline, econazole, efavirenz, enoxacin, erythromycin, sulfisoxazole, ethambutol, ethionamide, famciclovir, fluconazole, flucytosine, foscarnet, fosfomycin, furazolidone, ganciclovir, gentamicin, grepafloxacin, griseofulvin, halofantrine, hydroxychloroquine, imipenem; cilastatin, indinavir, ribavirin, iodoquinol, isoniazid, pyrazinamide, rifampin, isoproterenol, itraconazole, ivermectin, kanamycin, ketoconazole, lamivudine, zidovudine, levofloxacin, lincomycin, lindane, lomefloxacin, loracarbef, mebendazole, mefloquine, meropenem, metaproterenol, metronidazole, mezlocillin, miconazole, minocycline, nafcillin, naftidine, nalidixic acid, natamycin, nelfinavir, neomycin, netilmicin, nevirapine, nitrofurantoin, norfloxacin, nystatin, triamcinolone, ofloxacin, oxacillin, oxytetracycline, oxiconazole, paromomycin, aminosidine, penicillin G, penicillin V, pentamidine, permethrin, phenazopyridine, sulfisoxazole, piperacillin, piperacillin; tazobactam, praziquantel, primaquine, prochlorperazine, pyrazinamide, pyrimethamine, sulfadoxine, quinine, rifampin, rifapentine, rimantadine, ritonavir, saquinavir, sparfloxacin, spectinomycin, stavudine, sulconazole, sulfabenzamide; sulfacetamide; sulfathiazole, sulfacetamide, sulfacytine, sulfadiazine, sulfamethoxazole, trimethoprim, sulfanilamide, sulfasalazine, sulfisoxazole, terbinafine, terconazole, thiabendazole, ticarcillin, tioconazole, tobramycin, triacetin, triamcinolone, trimethoprim, trimetrexate, troleandomycin, trovafloxacin, alatrofloxacin, valacyclovir, vancomycin, zalcitabine and zidovudine.

The compositions of the invention can additionally include combinations of two or more pharmaceutically active compounds, such as two or more of the compounds listed above.

The pharmaceutically active compound preferably includes one or more cationic or anionic functional groups. Suitable cationic groups include primary, secondary and tertiary amino groups, imino groups, quaternary ammonium groups, amidino groups, guanidino groups, phosphonium groups, and sulfonium groups. Suitable anionic groups include carboxylate, sulfonate, phosphonate, sulfamate, sulfate ester, phosphate ester, sulfinate, phosphinate, carbonate, thiocarboxylate and carbamate groups. Preferred cationic groups include primary, secondary and tertiary amino groups, imino groups and quaternary ammonium groups. Preferred anionic groups include carboxylate and sulfonate groups. Preferably, the pharmaceutically active compound comprises two or more anionic groups or two or more cationic groups. In one embodiment, the pharmaceutically active compound comprises three or more anionic groups or three or more cationic groups.

Certain pharmaceutically active compounds contain both acidic groups and cationic groups and exist as zwitterions at physiological pH. Such compounds can, optionally, be present in the compositions of the invention in a modified, or prodrug, form in which one or more acidic functional groups are esterified. Such esterification increases the net positive charge of the compound. Similarly, the pharmaceutically active compound can have amino groups which have been acylated or sulfonylated to form an amide or sulfonamide, respectfully. Such acylation results in an increase in the net negative charge of the pharmaceutically active compound.

The ionic macromolecule used in the formulations of the invention may be a linear or cross-linked polymer comprising monomers which bear a positive or negative charge at a certain pH. In one embodiment, each of the monomeric units in the polymer comprises an acidic functional group or a basic functional group. In another embodiment, a fraction of the monomers within the polymer are functionalized with an acid functional group or a basic functional group. Preferably, the polymer comprises either anionic functional groups or cationic functional groups, although the polymer can comprise both cationic and anionic functional groups, so long as the proportion of these groups allows for the desired net ionic charge at the desired pH. Each of the cationic or anionic groups in the polymer can be the same or different, although in preferred embodiments they are the same.

In one embodiment, the polymer includes basic or cationic functional groups such as primary, secondary or tertiary amino groups, quaternary ammonium groups, guanidino groups, amidino groups, phosphonium groups or sulfonium groups. Preferably, the basic or cationic groups are primary, secondary or tertiary amino groups or quaternary ammonium groups.

In another embodiment, the polymer includes acidic or anionic functional groups, such as carboxylate, sulfonate, phosphonate, sulfate ester, phosphate ester, sulfamate or carbamate groups. Preferably the anionic groups are carboxyl groups. The ionic macromolecule is physiologically compatible and is, preferably, biodegradable or bioresorbable. Preferred ionic macromolecules are suitable for administration via intraperitoneal, intramuscular or intravenous injection or inhalation. Suitable ionic polymers include ionic polysaccharides; ionic polyesters; ionic polyamides, for example, ionic peptides; polyacrylates and polyamines. Examples of suitable ionic polymers include, but are not limited to, carboxymethylcellulose, poly(arginine), poly(lysine), poly(glutamic acid), poly(aspartic acid), poly(arginine-co-glycine), poly(lysine-co-glycine), poly(glutamic acid-co-glycine), poly(aspartic acid-co-glycine), poly(arginine-co-alanine), poly(lysine-co-alanine), poly(glutamic acid-co-alanine), poly(aspartic acid-co-alanine), diethylaminoethyldextran, diethylaminoethylcellulose, starch glycolate, polygalacturonic acid, poly-d-glucosamine (chitosan), poly(acrylic acid), poly(ethyleneimine), poly(allylamine), polyvinylamine, carrageenan, and alginic acid.

Preferred ionic polymers include ionic polysaccharides and ionic polypeptides. The ionic polymer can be linear or cross-linked. For example, the ionic polymer can be cross-linked to varying extents, using ionic cross-linking or covalent cross-linking. In one embodiment, the ionic polymer bears a net ionic charge and is cross-linked by the addition of an amount of an oppositely charged cross-linking polymer. The relative amounts of the two polymers can be varied to provide different degrees of cross-linking, but should be such that the combination retains a net ionic charge sufficient to bind a desired amount of the pharmaceutically active compound. For example, an anionic polymer, such as carboxymethylcellulose, can be cross-linked with varying amounts of a cationic polymer, such as poly(lysine), while a cationic polymer, such as diethylaminoethylcellulose can be cross-linked with an anionic polymer, such as poly(glutamic acid).

In another embodiment, the ionic polymer is covalently cross-linked. In one example, ionic polymers comprising carboxylate groups are cross-linked as is known in the art by reacting a fraction of the carboxylate groups, or activated derivatives thereof, with a suitable cross-linking reagent such as a dialcohol, an aminoalcohol or a diamine, under conditions suitable for forming ester and/or amide linkages. In this case, the ionic polymer will comprise carboxylate groups and ester/amide groups, with the ester/amide groups on one polymer strand linked to ester/amide groups on another polymer strand by bridging groups derived from the dialcohol, amino alcohol or diamine used. Preferably, the dialcohol, amino alcohol or diamine is pharmaceutically acceptable.

In another example, a cationic polymer comprising primary, secondary or tertiary amino groups can be cross-linked by reacting a fraction of the amino groups with a cross-linking reagent comprising two or more functional groups capable of reacting with an amino group to form a carbon-nitrogen bond. For example, the cationic polymer can be reacted with a dicarboxylate, disulfonate or activated derivative thereof, or a compound comprising two or more alkylating functional groups, such as 1,2-dihaloethane, epichlorohydrin and others known in the art. Such reactions result in a polymer in which a fraction of the amino nitrogen atoms in one polymer strand are connected to amino groups in other polymer strands via bridging groups derived from the cross-linking agent. When the nitrogen-carbon bond formed via cross-linking, such as an amide bond or a sulfonamide bond, is labile under physiological conditions, the cross-linking reagent is preferably physiologically acceptable.

The solid ionic complex can have a range of compositions. For example, the complex can comprise from about 2% pharmaceutically active compound to about 90% pharmaceutically active compound. The complex can comprise from about 98% ionic macromolecule to about 10% ionic macromolecule. Preferably, the solid ionic complex comprises 10% or greater, 20% or greater or 30% or greater pharmaceutically active compound. More preferably, the solid ionic complex comprises 40% or greater or 50% or greater pharmaceutically active compound. Preferably, the solid ionic complex comprises 90% or less; 80% or less; or 70% or less ionic macromolecule. More preferably, the solid ionic complex comprises 60% or less or 50% or less ionic macromolecule. All percentages disclosed herein are weight/weight unless otherwise indicated. Ranges of values using a combination of any of the above recited values as upper and/or lower limits are intended to be included.

The ratio (weight/weight) of the pharmaceutically active compound to the ionic macromolecule in the solid ionic complex of the invention is, preferably, about 10, 9, 8, 7, 6, 5, 4, 3, 2, 1, 0.75, 0.5, 0.25 or 0.1. Preferably the ratio of the pharmaceutically active compound to the ionic macromolecule is about 0.5, 0.75, 1 or greater.

In one embodiment, the solid ionic complex consists essentially of the ionic macromolecule and the pharmaceutically active compound. Typically, such a solid ionic complex will be hydrated and the mass of the complex will include some amount of water. The degree of hydration can be determined by subjecting the complex to dehydrating conditions, preferably conditions under which the pharmaceutically active compound and the ionic macromolecule are stable, and determining the resulting weight decrease.

In another embodiment, the solid ionic complex comprises a first pharmaceutically active compound, the ionic macromolecule and one or more additional substances. Suitable additional substances include a second pharmaceutically active compound, which, preferably, has a net charge at the desired pH which is of the same sign as that of the first pharmaceutically active compound. The additional substance or substances can also include one or more pharmaceutically acceptable excipients or other agents which modulate the properties of the complex, such as solubility.

The solid ionic complex is, preferably, substantially insoluble in aqueous solvent at the desired pH, e.g., physiological pH. The term “substantially insoluble” is used herein to refer to a material that has negligible solubility, e.g., in water, under a given set of conditions. It is to be understood that a substantially insoluble material can have finite solubility, but generally is soluble to an extent providing a concentration of pharmaceutically active compound no greater than 10 mM, 1 mM, 100 μM, 10 μM or 1 μM. For a given pharmaceutically active compound, the ionic macromolecule and additional exipients, if any, can be selected to optimize the properties of the solid ionic complex with respect to aqueous solubility and/or compound loading, among others. For example, the extent of cross-linking of the ionic macromolecule can be varied, with more extensive cross-linking expected to lead to less soluble complexes. Cross-linking can be accomplished using methods known in the art, such as covalent cross-linking or ionic cross-linking. Ionic cross-linking can be accomplished, for example, by including an amount of a polymer having at the desired pH a net ionic charge opposite in sign to that of the ionic macromolecule.

The solubility of a complex comprising an ionic macromolecule and an ionic pharmaceutically active compound can also be modulated by including an excipient such as a di- or tri-valent metal cation, such as Al³⁺, Ca²⁺ or Mg²⁺ or a polyvalent anion, such as phosphate, carbonate or sulfate. One of skill in the art can readily determine a combination of excipients, cross-linking agents and extent of cross-linking to provide a complex having the desired solubility.

The present invention further includes pharmaceutical compositions comprising a solid ionic complex of a pharmaceutically active compound and an ionic macromolecule and a pharmaceutically acceptable carrier. For example, the solid ionic complex can be suspended in a vehicle suitable for injection.

Pharmaceutical compositions of the invention suitable for injectable use may include physiological saline, bacteriostatic water, Cremophor EL™ (BASF, Parsippany, N.J.) or phosphate buffered saline (PBS). In all cases, the composition must be sterile and should be fluid to the extent necessary for easy syringability to exist. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the pharmaceutical composition an agent which delays absorption, for example, aluminum monostearate and gelatin.

The pharmaceutical composition can also include the solid ionic complex and a carrier suitable for administration via inhalation. Particular compositions suitable for inhalation include dry powders, liquid solutions or suspensions suitable for nebulization, and propellant formulations suitable for use in metered dose inhalers (MDI's). Suitable carriers for inhalation include dry bulking powders, such as sucrose, lactose, trehalose, human serum albumin (HSA), and glycine. Other suitable dry bulking powders include cellobiose, dextrans, maltotriose, pectin, sodium citrate, sodium ascorbate and mannitol.

The solid ionic complex can also be suspended in a suitable aerosol propellant, such as a chlorofluorocarbon (CFC) or a hydrofluorocarbon (HFC). Suitable CFC's include trichloromonofluoromethane (propellant 11), dichlorotetrafluoromethane (propellant 114), and dichlorodifluoromethane (propellant 12). Suitable HFC's include tetrafluoroethane (HFC-134a) and heptafluoropropane (HFC-227). Preferably, for incorporation into the aerosol propellant, the solid ionic complex of the present invention can be processed into respirable particles. The particles are then suspended in the propellant, and, optionally, coated with a surfactant to enhance their dispersion. Suitable surfactants include oleic acid, sorbitan trioleate, and various long chain diglycerides and phospholipids. The inhalable compositions of the invention can be administered using a conventional dry powder inhaler, nebulizer or metered dose inhaler.

Preparation of the Pharmaceutical Compositions

The present invention also relates to a method of preparing a solid ionic complex comprising an ionic macromolecule and a pharmaceutically active compound. The solid ionic complex of the invention is prepared by combining the pharmaceutically active compound and the carrier macromolecule under conditions such that a water-insoluble complex of the pharmaceutically active compound and the ionic carrier macromolecule forms. In one embodiment, the method includes providing a pharmaceutically active compound and an ionic carrier macromolecule; and combining the pharmaceutically active compound and the carrier macromolecule under conditions such that a water-insoluble complex of the pharmaceutically active compound and the carrier macromolecule forms.

The ionic macromolecule can be combined with the pharmaceutically active compound in a variety of ways. For example, a solution of the ionic macromolecule can be mixed with a solution of the pharmaceutically active compound under conditions suitable for precipitation of the ionic complex. The two solutions can include the same solvent or different solvents. Preferably, if the solvents are different, they are miscible. The ionic macromolecule can be added as a solid to a solution of the pharmaceutically active compound or the pharmaceutically active compound can be added to a solution of the ionic macromolecule.

In another embodiment, the ionic macromolecule and the pharmaceutically active compound are added to a solvent in which neither is substantially soluble, but in which a by-product of the complexation, or ion-exchange process, is expected to be soluble. For example, a pharmaceutically active compound having a water-insoluble hydrochloride salt can be added to an aqueous suspension of the sodium salt of an ionic macromolecule. The resulting suspension can be agitated for a sufficient period of time for formation of the desired solid ionic complex. In this case, the ion exchange process resulting in the desired solid ionic complex is driven, at least in part, by the solubility of the sodium chloride product.

Once the solid ionic complex precipitates, the precipitate can be removed from the solution by means known in the art, such as filtration (e.g., through a 0.45 micron nylon membrane), centrifugation and the like. The recovered paste then can be dried (e.g., in vacuo or in a 70° C. oven) and the solid can be milled or pulverized to a powder by means known in the art (e.g., hammer or gore milling, or grinding in mortar and pestle). Following milling or pulverizing, the powder can be sieved through a screen (preferably a 90 micron screen) to obtain a uniform distribution of particles. Moreover, the recovered paste can be frozen and lyophilized to dryness.

The powder form of the complex can be dispersed in a carrier solution to form a liquid suspension or semi-solid dispersion suitable for injection. Accordingly, in various embodiments, a pharmaceutical formulation of the invention is a dry solid, a liquid suspension or a semi-solid dispersion. Examples of liquid carriers suitable for use in liquid suspensions include saline solutions, glycerin solutions, lecithin solutions and oils suitable for injection.

In another embodiment, the pharmaceutical formulation of the invention is a sterile formulation. For example, following formation of the water-insoluble complex, the complex can be sterilized, optimally by gamma irradiation or electron beam sterilization. Accordingly, the method of the invention for preparing a pharmaceutical formulation described above can further comprise sterilizing the water-insoluble complex by gamma irradiation or electron beam irradiation. Preferably, the formulation is sterilized by gamma irradiation using a gamma irradiation dose of at least 15 KGy. In other embodiments, the formulation is sterilized by gamma irradiation using a gamma irradiation dose of at least 19 KGy or at least 24 KGy. Alternatively, to prepare a sterile pharmaceutical formulation, the water-insoluble complex can be isolated using conventional sterile techniques (e.g., using sterile starting materials and carrying out the production process aseptically). Accordingly, in another embodiment of the method for preparing a pharmaceutical formulation described above, the water-insoluble complex is formed using aseptic procedures.

Use of the Pharmaceutical Compositions

In another embodiment, the present invention provides a method for treating a subject for a condition treatable with a pharmaceutically active compound. The method includes administering to the subject the pharmaceutical compositions of the invention in an amount effective to treat the condition.

The subject can be any animal in need of treatment for which the pharmaceutically active compound is indicated, and is preferably a mammal, such as a canine, feline, bovine, equine, ovine or porcine animal or a primate, such as a monkey, an ape or a human. More preferably, the subject is a human.

In one embodiment, the subject is injected with the pharmaceutical composition using methods known in the art. The injection may be an intravenous, intramuscular, subcutaneous or intraparenteral injection.

In another embodiment, the subject is caused to inhale the composition using means which are known in the art, including the use of a dry powder inhaler, nebulizer or metered dose inhaler.

Devices which can be used to administer the pharmaceutical compositions of the invention are also contemplated. Examples include a syringe which houses a pharmaceutical composition comprising a solid ionic complex comprising the pharmaceutically active compound and an ionic bioerodable macromolecule, where the complex is suspended in a vehicle suitable for injection, and an inhalation device which houses a pharmaceutical composition comprising a solid ionic complex comprising the pharmaceutically active compound and an ionic bioerodable macromolecule and a carrier suitable for inhalation. The inhalation device can be, for example, a dry powder inhaler, a nebulizer or a metered dose inhaler.

Screening Assays

The invention also provides screening methods for identifying pharmaceutically active compounds which can form an insoluble complex with an ionic polymer, or, for a given pharmaceutically active compound, the particular ionic polymer and/or other conditions which favor the formation of such a complex. In one embodiment, the method comprises the steps of (1) providing a multiplicity of solutions, each solution comprising a pharmaceutically active compound; (2) contacting the solutions of step (1) with a solution comprising an ionic polymer to produce a mixture comprising a pharmaceutically active compound and an ionic polymer and (3) determining the turbidity of the mixture of step (2).

In another embodiment, the invention provides a method for selecting an ionic polymer which will form an insoluble complex with a particular pharmaceutically active compound. The method comprises the steps of (1) providing a solution comprising the pharmaceutically active compound; (2) providing n distinct solutions, where n is an integer of two or greater, each solution comprising an ionic polymer; (3) contacting each of n aliquots of the solution comprising the pharmaceutically active compound with one of the solutions comprising an ionic polymer, thereby forming n mixtures comprising a pharmaceutically active compound and an ionic polymer; and (4) determining the turbidity of each mixture of step (3).

The n distinct solutions comprising an ionic polymer differ each from the others in terms of at least one parameter of interest. Possible parameters of interest include identity of the ionic polymer; average molecular weight of the ionic polymer; molecular weight dispersity of the ionic polymer; concentration of the ionic polymer; degree of substitution of the ionic polymer; pH; ionic strength; temperature; presence/absence and others that will be recognized by one of skill in the art.

The turbidity of a mixture can be determined using a variety of means known in the art. For example, if the degree of turbidity is great enough, it may be detectable by the naked eye. Preferably, the turbidity is determined quantitatively as the extent of light scattering at a particular wavelength. For example, the percent transmittance or apparent absorbance of light at a given wavelength can be measured and compared to a standard, such as water, or the solution of the pharmaceutically active compound or the solution of the ionic polymer. Preferably, the wavelength used is a wavelength at which neither the pharmaceutically active compound nor the ionic polymer absorb significantly. An increase in apparent absorbance (decrease in percent transmittance) compared to the blank is indicative of the formation of a solid phase dispersed in the solution.

The methods of the invention are preferably performed in a format which facilitates the rapid evaluation of a large number of conditions, ionic polymers and/or drugs. For example, the mixtures can be formed in the wells of a 96 well plate and the turbidity can be measured by determining the apparent absorbance at a suitable wavelength using a plate reader.

The invention is further illustrated by the following examples, which should not be construed as further limiting. The contents of all references, pending patent applications and published patents, cited throughout this application, as well as the Sequence Listing are hereby expressly incorporated by reference.

### EXAMPLES

### Example 1

### Screen For Compounds which Form Insoluble Complexes with Ionic Polymer

A series of pharmaceutically active compounds were chosen having a variety of structures. Carboxymethylcellulose sodium (“CMC”) solutions were prepared by making serial dilutions from a CMC stock solution prepared by dissolving 14.10 g of CMC in 500 mL water. After correcting for the nominal 84.5% purity of the CMC, the CMC concentration of the solution was 20 mg/mL. Dilutions of this stock solution were used to prepare solutions having CMC concentrations of 0.05, 0.08, 0.1, 0.5, 5, 10 and 20 mg/mL. These seven solutions were further subdivided into three fractions each. The pH of the first fraction was measured, and this fraction was not modified. The pH of the second fraction was adjusted to about pH 6 with acetic acid. The pH of the third fraction was adjusted to about pH 5 with acetic acid.

Drug solutions were prepared by adding a known amount of drug to conical polypropylene centrifuge tubes and adding water, ethanol or dimethyl sulfoxide to dissolve the drug. Certain of the drugs dissolved readily to provide a homogeneous solution, while others were incompletely dissolved and the resulting mixtures were filtered to yield a homogeneous solution. In cases in which not all of the drug dissolved, the concentration is indicated as less than the concentration which would have resulted had all drug dissolved.

Drug and CMC solutions were mixed in microtiter plates. In general, 100 μL CMC solution was added to a well, followed by 100 μL drug solution. The plate was then agitated at the maximum speed provided by the plate reader, and the absorbance of each well at 450 nm was then measured. For each plate, water blanks, negative controls (ipratropium bromide+CMC; water+CMC; water+drug solution) and positive controls (octreotide+CMC) were included. Each drug solution was mixed with the ca. pH 7 CMC solutions at room temperature, 35° C. and 50° C.; and with the pH 6 and pH 5 solutions at room temperature. The turbidity was measured immediately after mixing and after an additional one hour.

Results

The overall results of this study are presented in Table I, in which each compound examined is classified into one of three groups: (1) compounds which form a complex with CMC; (2) compounds which do not form a complex with CMC under the conditions of this study; and (3) compounds having properties which are incompatible with the screen. Category 3 includes, for example, compounds which absorb at 450 nm and compounds which were initially solubilized in non-aqueous media and precipitated when mixed with water.

TABLE I

| Compounds which | Compounds which | |
|---|---|---|
| formed a complex | did not form complex | Exceptions |
| Bendroflumethiazide | Acyclovir | Dimenhydrinate |
| Bisacodyl | Carbetapentane | Haloperidol |
| Carbidopa | Isoniazid | Nifedipine |
| Chlorothiazide | Isoproterenol | Pimozide |
| Chlorthalidone | Pyrilamine | Rifampicin |
| Cimetidine | Ribavirin | triamterene |
| Cinoxacin | Sulfacetamide | |
| Droperidol | Sulfadiazine | |
| Furazolidone | Sulfathiazole | |
| Guanabenz | | |
| Lidocaine | | |
| Loxapine | | |
| Minoxidil | | |
| Ofloxacin | | |
| Perphenazine | | |
| Phenyltoloxamine | | |
| Physostigmine | | |
| Quinidine | | |
| Sulfabenzamide | | |
| Thiothixene | | |

Equivalents

Those skilled in the art will recognize, or be able to ascertain using no more than routine experimentation, many equivalents to the specific embodiments of the invention described herein. Such equivalents are intended to be encompassed by the following claims.

1. A pharmaceutical composition comprising a solid ionic complex, said complex comprising a pharmaceutically active compound and an ionic macromolecule.

2. The pharmaceutical composition of claim 1 wherein the pharmaceutically active compound has a net positive charge.

3. The pharmaceutical composition of claim 2, wherein the pharmaceutically active compound has at least one functional group selected from the group consisting of primary amino groups, secondary amino groups, tertiary amino groups, imino groups, quaternary ammonium groups, amidino groups, guanidino groups, phosphonium groups and sulfonium groups.

4. The pharmaceutical composition of claim 1, wherein the pharmaceutically active compound has a net negative charge.

5. The pharmaceutical composition of claim 4, wherein the pharmaceutically active compound has at least one functional group selected from the group consisting of carboxylate groups, sulfonate groups, phosphonate groups, sulfamate groups, sulfate ester groups, phosphate ester groups, sulfinate groups, phosphinate groups, carbonate groups, thiocarboxylate groups and carbamate groups.

6. The pharmaceutical composition of claim 2, wherein the pharmaceutically active compound contains at least one functional group selected from the group consisting of primary amino groups, secondary amino groups, tertiary amino groups, imino groups and quaternary ammonium groups.

7. The pharmaceutical composition of claim 2, wherein the pharmaceutically active compound has at least one functional group selected from the group consisting of carboxylate and sulfonate.

8. The pharmaceutical composition of claim 1, wherein the pharmaceutically active compound has a molecular weight of about 1000 amu or less.

9. The pharmaceutical composition of claim 1, wherein the pharmaceutically active compound has a molecular weight of about 750 amu or less.

10. The pharmaceutical composition of claim 1, wherein the pharmaceutically active compound has a molecular weight of about 500 amu or less.

11. The pharmaceutical composition of claim 2, wherein the pharmaceutically active compound has a net charge of at least +1.

12. The pharmaceutical composition of claim 2, wherein the pharmaceutically active compound has a net charge of at least +2.

13. The pharmaceutical composition of claim 4, wherein the pharmaceutically active compound has a net charge of at least −1.

14. The pharmaceutical composition of claim 4, wherein the pharmaceutically active compound has a net charge of at least −2.

15. The pharmaceutical composition of claim 1, wherein the ionic macromolecule comprises at least one functional group selected from the group consisting of carboxylic acid, sulfonic acid, sulfamic acid, primary amine, secondary amine, tertiary amine, quaternary ammonium, guanidino and amidino.

16. The pharmaceutical composition of claim 4, wherein the ionic macromolecule is a polypeptide or a polysaccharide.

17. The pharmaceutical composition of claim 1, wherein a single dose of the solid ionic complex provides sustained delivery of the pharmaceutically active compound to a subject for at least one week after the pharmaceutical composition is administered to the subject.

18. The pharmaceutical composition of claim 1, wherein a single dose of the solid ionic complex provides sustained delivery of the pharmaceutically active compound to a subject for at least two weeks after the pharmaceutical composition is administered to the subject.

19. The pharmaceutical composition of claim 1, wherein a single dose of the solid ionic complex provides sustained delivery of the pharmaceutically active compound to a subject for at least three weeks after the pharmaceutical composition is administered to the subject.

20. The pharmaceutical composition of claim 1, wherein a single dose of the water-insoluble complex provides sustained delivery of the pharmaceutically active peptide to a subject for at least four weeks after the pharmaceutical composition is administered to the subject.

21. The pharmaceutical composition of claim 1, wherein said solid ionic complex is a lyophilized solid.

22. The pharmaceutical composition of claim 1, wherein said solid ionic complex is suspended as a liquid suspension or dispersed as a semi-solid dispersion.

23. The pharmaceutical composition of claim 1, wherein the pharmaceutically active compound content of the solid ionic complex is at least 50% by weight.

24. The pharmaceutical composition of claim 1, wherein the pharmaceutically active compound content of the solid ionic complex is at least 60% by weight.

25. The pharmaceutical composition of claim 1, wherein the pharmaceutically active compound content of the solid ionic complex is at least 70% by weight.

26. The pharmaceutical composition of claim 1, wherein the pharmaceutically active compound content of the solid ionic complex is 50% to 90% by weight.

27. The pharmaceutical composition of claim 1, wherein the pharmaceutically active compound and the ionic macromolecule used to form the solid ionic complex are combined at a weight ratio of ionic macromolecule:pharmaceutically active compound of 0.5:1 to 0.1:1

28. The pharmaceutical composition of claim 1, wherein the pharmaceutically active compound and the ionic macromolecule used to form the solid ionic complex are combined at a weight ratio of ionic macromolecule:pharmaceutically active compound of 1:1 to 0.1:1

29. The pharmaceutical composition of claim 1, wherein the solid ionic complex is not a microcapsule.

30. A packaged formulation for treating a subject for a condition treatable with a pharmaceutically active compound, comprising the pharmaceutical composition of claim 1 packaged with instructions for using the composition for treating a subject having a condition treatable with a pharmaceutically active compound.

31. A method for treating a subject for a condition treatable with a pharmaceutically active compound, comprising administering to the subject the pharmaceutical composition of claim 1.

32. A method for preparing a pharmaceutical formulation, comprising:
providing a pharmaceutically active compound and an ionic macromolecule;
combining the pharmaceutically active compound and the ionic macromolecule under conditions such that a solid ionic complex of the pharmaceutically active compound and the ionic macromolecule forms; and
preparing a pharmaceutical formulation comprising the solid ionic complex.

33. The method of claim 32, wherein a solution of the pharmaceutically active compound and a solution of the ionic macromolecule are combined until a water-insoluble complex of the pharmaceutically active compound and the ionic macromolecule precipitates.

34. The method of claim 33, wherein the solution of the pharmaceutically active compound and the solution of the ionic macromolecule are aqueous solutions.

35. The method of claim 33, wherein the solution of the pharmaceutically active compound and the solution of the ionic macromolecule are combined and heated until a water-insoluble complex of the pharmaceutically active compound and the ionic macromolecule precipitates.

36. The method of claim 33, further comprising sterilizing the water-insoluble complex by gamma irradiation or electron beam irradiation.

37. The method of claim 33, wherein the water-insoluble complex is formed using aseptic procedures.

38. A method for identifying a pharmaceutically active compound which can form an insoluble complex with an ionic polymer, the method comprising:
(1) providing a multiplicity of solutions, each of said solutions comprising a pharmaceutically active compound;
(2) contacting the solutions of step (1) with an ionic polymer to produce a mixture comprising a pharmaceutically active compound and an ionic polymer; and
(3) determining the turbidity of the mixture of step (2), thereby identifying a pharmaceutically active compound which can form an insoluble complex with an ionic polymer.

39. A method for selecting an ionic polymer capable of forming an insoluble complex with a pharmaceutically active compound, the method comprising:
(1) providing a solution comprising the pharmaceutically active compound;
(2) providing n distinct solutions, where n is an integer of two or greater, each of said n distinct solutions comprising an ionic polymer;
(3) contacting each of n aliquots of the solution comprising the pharmaceutically active compound with one of the solutions comprising an ionic polymer, thereby forming n mixtures comprising a pharmaceutically active compound and an ionic polymer; and
(4) determining the turbidity of each mixture of step (3), thereby selecting an ionic polymer capable of forming an insoluble complex with a pharmaceutically active compound.
